(19) United States
(12) Patent Application Publication
(10) Pub. No.: US 2009/0104128 A1
(43) Pub. Date: Apr. 23, 2009

(54) Denture adhesive compositions with anti-ucler agents

(75) Inventor: Jeffrey T. Haley, Mercer Island, WA (US)

Correspondence Address: Jeffrey T. Haley, 13434 SE 27th Place, Bellevue, WA, 98005, US

(73) Assignee: Orahealth Corporation, Bellevue, WA (US)

(21) Appl. No.: 12/287,646

(22) Filed: Oct. 10, 2008

Related U.S. Application Data
(60) Provisional application No. 60/999,516, filed on Oct. 17, 2007.

(51) Int. Cl. A61K 8/97

(52) U.S. Cl. 424/58

(57) ABSTRACT

The present invention relates to methods and compositions for reducing, inhibiting, and/or preventing denture sores, comprising a denture adhesive component and an effective amount of an anti-ulcer agent selected from the group consisting of Glycyrrhiza extract (GX), xylitol, and cobalamin. A denture wearer in need thereof applies the composition to the oral cavity and/or the denture prosthesis at a spot where a denture sore tends to form and thereafter secures the denture to the oral cavity.

This application claims priority from U.S. provisional application 60/999516 filed Oct. 17, 2007

### TECHNICAL FIELD

The present invention relates to compositions and methods for reducing, inhibiting, preventing, and/or reducing pain from denture sores, comprising a denture adhesive component and an effective amount of an anti-ulcer agent selected from the group consisting of Glycyrrhiza extract (GX), xylitol, and cobalamin. These compositions provide the above benefits at the points where denture contact causes sores without interfering with denture hold.

### BACKGROUND OF THE INVENTION

Ordinary removable dentures, dental plates and the like, comprise teeth mounted in a suitable plate or base. Many denture wearers experience “denture sores”, an ulcer that occurs at a point of contact between the denture and the gums or mucosa. Agents known to reduce, inhibit, prevent and/or reduce pain from mouth ulcers generally, particularly ulcers not caused by dentures, include Glycyrrhiza (licorice root) extract (GX), including refined glycyrrhizin and glycyrrhetinic acid, xylitol, and cobalamin in its various forms, including cyanocobalamin, hydroxocobalamin, and methylcobalamin.

Denture adhesives are used to fill interstices between dentures and gums or tissues. Prior to placement of the denture in the oral cavity, a denture adhesive is applied to the denture-plate surface which, for a perfect fit, should uniformly contact the gums and mucous tissues. The denture adhesive is formulated not only for its adherent properties, but also to provide a cushion or gasket between the denture and the gums or tissues, thereby positioning the denture securely in the oral cavity.

Considerable effort has been made over the years to develop improved denture adhesive compositions. Both synthetic and natural polymers and gums have been used alone, in combination, and in combination with various other adhesive materials in an attempt to improve hold and reduce oozing of the adhesive from under the dental plate. For example, alkyl vinyl ether-maleic copolymers and salts thereof are known for providing good hold in denture adhesive compositions. Such disclosures include: U.S. Pat. No. 3,003,988, Germann et al., issued Oct. 10, 1961; U.S. Pat. No. 4,980,391, Kumar et al., issued Dec. 25, 1990; U.S. Pat. No. 5,073,604, Holeva et al., issued Dec. 17, 1991; U.S. Pat. No. 5,525,652, Clarke, issued Jun. 11, 1996; U.S. Pat. No. 5,340,918, Kittrell et al., issued Aug. 23, 1994; U.S. Pat. No. 5,830,933, Synodis et al., issued Nov. 3, 1998; and U.S. Pat. No. 6,475,498, Rajaiah et al. issued Nov. 5, 2002.

In addition, strip/wafer type denture adhesives are also known. For example, U.S. Pat. No. 4,880,702 to Homan et al., issued Nov. 14, 1989 discloses a denture adhesive in the form of a strip consisting of three layers. EP 353,375, Altwirth, published Feb. 7, 1990, discloses an adhesive insert for dentures consisting of a adhesive permeated fibrous fleece and an adhesive consisting of a pasty mixture of alginate and/or carboxymethylcellulose, polyvinyl acetate and an alcoholic solvent.

### SUMMARY OF THE INVENTION

The present invention relates to compositions and methods for reducing, inhibiting, preventing, and/or reducing pain from denture sores by combining a denture adhesive component and an effective amount of an anti-ulcer agent selected from the group consisting of Glycyrrhiza extract (GX), xylitol, and cobalamin. A denture wearer in need thereof applies the composition to the oral cavity and/or the denture prosthesis at a spot where a denture sore tends to form and thereafter secures the denture to the oral cavity.

In one aspect, the invention is a denture adhesive composition that combats denture sores, comprising at least 15% of a denture adhesive mixture and an effective amount of Glycyrrhiza extract. The Glycyrrhiza extract may be a complex extract resulting from an extraction with a solvent including water and/or alcohol. The Glycyrrhiza extract may be a refined extract consisting of at least 50% glycyrrhizin and/or glycyrrhetinic acid.

In another aspect, the invention is a denture adhesive composition that combats denture sores, comprising at least 15% of a denture adhesive mixture and an effective amount of xylitol.

In another aspect, the invention is a denture adhesive composition that combats denture sores, comprising at least 15% of a denture adhesive mixture and an effective amount of cobalamin. The cobalamin may be in the form of one or more of cyanocobalamin, hydroxocobalamin, and methylcobalamin. Methylcobalamin is preferred.

In another aspect, the invention is a method of combating denture sores in the mouth of a denture wearer in need thereof, by mixing an anti-ulcer agent with a denture adhesive composition comprising at least 15% of the mixture and providing the mixture to the denture wearer with instructions to use for denture sores. The anti-ulcer agent may be an effective amount of at least one of: Glycyrrhiza extract, xylitol, and cobalamin.

In another aspect, the invention is a method of combating denture sores in the mouth of a denture wearer in need thereof, by applying a mixture of an anti-ulcer agent with a denture adhesive composition comprising at least 15% of the mixture to at least one of the denture or the mucosa and then securing the denture to the oral cavity. The anti-ulcer agent may comprise an effective amount of at least one of: Glycyrrhiza extract, xylitol, and cobalamin.

### DETAILED DESCRIPTION OF THE DRAWINGS

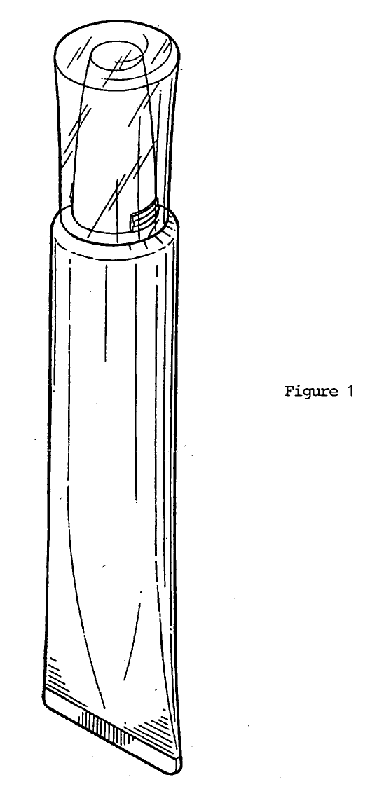

FIG. 1 shows a tube containing denture adhesive.

### DETAILED DESCRIPTION OF THE INVENTION

### Denture Adhesive Component

The present invention comprises a typical denture adhesive component, generally at a level of from about 15% to almost 100% by weight of the composition.

The denture adhesive component may be selected from the group consisting of AVE/MA, salts of AVE/MA, AVE/MA/IB, salts of AVE/MA/IB, and mixtures thereof, or any other effective denture adhesive. The term “AVE/MA” as used herein refers to alkyl vinyl ether-maleic acid or anhydride copolymer. The term “AVE/MA/IB” refers to terpolymers with alkyl vinyl ether, maleic acid or anhydride, and isobutylene. The term “mixed polymer salts” or “mixed salts”, as used herein, refers to salts of AVE/MA and/or AVE/MA/IB.

The AVE/MA copolymers have a range of specific viscosities. For example, the specific viscosity is preferably from 1.2 to 14, as preferably measured as a 1% weight/volume solution of the starting anhydride or acid of the copolymer, in methyl ethyl ketone at 25 degrees C. Other methods and solvents can be used to measure the specific viscosity such as a 1% weight/volume solution in DMF (dimethyl formamide) at 25 degrees C. and a 1% weight/volume solution in 2-butanone at 25 degrees C.

AVE/MA and salts thereof and AVE/MA/IB and salts thereof, are also described in U.S. Pat. No. 5,073,604 to Holeva et al., issued Nov. 17, 1991; U.S. Pat. No. 5,525,652, issued Jun. 11, 1996, Clarke et al.; U.S. Pat. No. 4,758,630, issued Jul. 19, 1988, Shah et al.; U.S. Pat. No. 5,304,616, issued Apr. 19, 1994, Rajaiah et al.; U.S. Pat. No. 5,424,058, issued Jun. 13, 1995, Rajaiah; U.S. Pat. No. 5,424,058, issued Jun. 13, 1995, Rajaiah et al.; U.S. Pat. No. 4,758,630, issued Jul. 19, 1988, Shah et al.; U.S. Pat. No. 5,830,933, issued Nov. 3, 1998, Synodis et al.; U.S. Pat. No. 2,047,398, issued Jul. 14, 1936, Voss et al.; U.S. Pat. No. 3,003,988, issued Oct. 10, 1961, Germann et al.; U.S. Pat. No. 5,880,172, Rajaiah et al., issued Mar. 9, 1999; U.S. Pat. No. 5,900,470, Prosise et al., issued May 4, 1999; U.S. Pat. No. 5,037,924, Tazi et al., issued Aug. 6, 1991; U.S. Pat. No. 5,082,913, Tazi et al, issued Jan. 21, 1992; U.S. Pat. No. 6,239,191, issued May 29, 2001; all of which are incorporated herein by reference in their entirety.

Suitable AVE/MA copolymers may be prepared by well-known methods of the prior art; see, for example, U.S. Pat. No. 2,782,182, and U.S. Pat. No. 2,047,398, both of which are incorporated by reference herein in their entirety. The terpolymers can be made by the methods discussed in U.S. Pat. No. 5,900,470, Prosise et al., issued May 4, 1999; U.S. Pat. No. 5,037,924, Tazi et al., issued Aug. 6, 1991; and U.S. Pat. No. 5,082,913, Tazi et al., issued Jan. 21, 1992, herein incorporated by reference in their entirety.

The alkyl vinyl ether maleic anhydride copolymers are obtained by co-polymerizing an alkyl vinyl ether monomer, such as methyl vinyl ether, ethyl vinyl ether, divinyl ether, propyl vinyl ether and isobutyl vinyl ether, with maleic anhydride to yield the corresponding alkyl vinyl ether-maleic anhydride copolymer which is readily hydrolyzable to the acid copolymer. Both anhydride and acid forms are also available from commercial suppliers. For example, the ISP Corporation, Wayne, N.J. provides both the polymeric free acid form (I) and the corresponding anhydride form under its “GANTREZ” trademark as the “GANTREZ S Series” and “GANTREZ AN Series”, respectively.

Methods of making mixed salts of AVE/MA polymers are further disclosed in U.S. Pat. No. 5,073,604, Holeva et al., issued Dec. 17, 1991; U.S. Pat. No. 5,872,161, Liang et al., issued Feb. 16, 1999; U.S. Pat. No. 5,830,933, Synodis et al., issued Nov. 3, 1998, all of which are herein incorporated by reference in their entirety.

The mixed salt polymers have exceptional adhesive qualities when contacted with water or saliva such that they are extremely useful as denture adhesive materials in denture compositions.

Alternative suitable adhesive components may include a water-soluble hydrophilic colloid or polymer having the property of swelling upon exposure to moisture to form a mucilaginous mass. In one embodiment the other adhesive components are selected from the group consisting of: natural gums, synthetic polymeric gums, synthetic polymers, mucoadhesive polymers, hydrophilic polymers, saccharide derivatives, cellulose derivatives, and mixtures thereof. In another embodiment the other adhesive components are selected from the group consisting of: natural gums, synthetic polymeric gums, anhydride, cellulose derivatives, and mixtures thereof. Examples of such materials include karaya gum, acacia gum, guar gum, gelatin, algin, sodium alginate, tragacanth, chitosan, polyethylene glycol, acrylamide polymers, cross-linked polyacrylic acid, polyvinyl alcohol, polyamines, polyquarternary compounds, polybutenes, silicones, ethylene oxide polymers, polyvinylpyrrolidone, cationic polyacrylamide polymers, and mixtures thereof.

In one embodiment these other adhesives are selected from the group consisting of polyethylene glycol, polyethylene oxide, karaya gum, sodium alginate, chitosan, polyvinyl alcohol, and mixtures thereof. In another embodiment these other adhesives are selected from the group consisting of cellulose derivatives such as methylcellulose, carboxymethylcellulose, hydroxyethylcellulose, hydroxypropylcellulose, hydroxypropylmethylcellulose, and mixtures thereof.

### The Anti-Ulcer Agent

The present compositions comprise an effective amount of an anti-ulcer agent. In one embodiment, the anti-ulcer agent is Glycyrrhiza extract. The Glycyrrhiza extract may be a complex extract resulting from an extraction with a solvent including water and/or alcohol. When a complex extract is used, the level may be between 10% and 75%. The Glycyrrhiza extract may be a refined extract consisting of at least 50% glycyrrhizin and/or glycyrrhetinic acid. In this case, the level will be between 3% and 60%.

In another embodiment, the anti-ulcer agent is xylitol at a level will be between 15% and 85%.

In another embodiment, the anti-ulcer agent is cobalamin. The cobalamin may be in the form of one or more of cyanocobalamin, hydroxocobalamin, and methylcobalamin. Methylcobalamin is preferred as tests have shown it to be superior for treatment of mouth ulcers. The level of cobalamin is generally from about 0.001% to about 10% by weight of the composition, to deliver between 50 and 500 micrograms with each application.

In another embodiment, the composition comprises any two of the above agents or all three of the above agents. An antimicrobial agent may be included. It should also be essentially compatible with the other components of the composition.

Once release begins, it is sustained for at least about 1 hour, in another embodiment for at least about 4 hours, in another embodiment from about 2 hours to about 4 hours, in yet another embodiment from about 4 hours to about 6 hours, and in yet another embodiment from about 6 hours to about 8 hours or longer.

### Plasticizers

In addition, one or more toxicologically-acceptable plasticizers may also be included in the present compositions. The term “toxicologically-acceptable”, as used herein, is used to describe materials that are suitable in their toxicity profile for administration to humans and/or lower animals. Plasticizers that may be used in the present compositions include dimethyl phthalate, diethyl phthalate, dioctyl phthalate, glycerin, diethylene glycol, triethylene glycol, sorbitol, tricresyl phosphate, dimethyl sebacate, ethyl glycolate, ethylphthalyl ethyl glycolate, o- and p-toluene ethyl sulfonamide, and mixtures thereof. Plasticizers may be present at a level of from about 1% to about 50%, preferably from about 2% to about 30%, by weight of the compositions.

### Flavors, Fragrance, Sensates

The compositions of the present invention may also include one or more components which provide flavor, fragrance, and/or sensate benefit (warming or cooling agents). Suitable components include natural or artificial sweetening agents, menthol, menthyl lactate, wintergreen oil, peppermint oil, spearmint oil, leaf alcohol, clove bud oil, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof, as well as coolants.

The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat.RTM. manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosure of both are herein incorporated by reference in their entirety. These agents may be present at a level of from about 0% to about 50%, by weight of the composition.

### Other Active Ingredients

The present adhesive compositions can be used to deliver one or more therapeutic actives suitable for topical administration to mucosal or wet tissues. The phrase “therapeutic actives”, as used herein, describes agents which are pharmacologically active when absorbed through wet tissue or mucosal surfaces of the body such as the oral cavity. Therapeutic actives may be present at a level of from about 0% to about 50%, by weight of the composition.

Therapeutic actives that are useful in the present compositions include anti-inflammatory agents such as aspirin, acetaminophen, naproxen and its salts, ibuprofen, ketorolac, flurbiprofen, indomethacin, eugenol, or hydrocortisone; anti-biotic and anti-microbial agents; anesthetic agents such as lidocaine or benzocaine; anti-fungals; aromatics such as camphor, eucalyptus oil, aldehyde derivatives such as benzaldehyde; and steroids. It is recognized that in certain forms of therapy, combinations of these agents in a single delivery system may be useful in order to obtain an optimal effect.

### Other Optional Ingredients

Other suitable ingredients include colorants, preservatives such as methyl and propyl parabens; and thickeners such as silicon dixode and polyethylene glycol. Colorants, preservatives, and thickeners may be present at levels of from about 0% to about 20%, preferably from about 1% to about 10%, by weight of the composition.

### Process for Preparation of the Composition

A process for preparing denture adhesive compositions of the present invention (creams, powders, wafers, liquids, aerosols, pastes) comprises conventional methods disclosed in the art. Conventional methods are taught in U.S. Pat. No. 5,525,652, issued Jun. 11, 1996, Clarke et al.; U.S. Pat. No. 3,003,988, issued Oct. 10, 1961, Germann et al.; U.S. Pat. No. 5,073,604, Holeva et al., issued Dec. 17, 1991; and U.S. Pat. No. 5,872,161, Liang et al., issued Feb. 16, 1999, all of which are herein incorporated by reference in their entirety.

A process for the preparation of the present denture adhesive compositions optionally comprising a non-adhesive self-supporting layer, comprises coating a weighed amount of the adhesive components onto the non-adhesive self-supporting layer. This process is disclosed in U.S. Pat. No. 5,877,233, Liang et al, issued Mar. 2, 1999; U.S. Pat. No. 5,872,160, issued Feb. 16, 1999, Liang et al.; U.S. Pat. No. 5,880,172, Rajaiah et al., filed Oct. 25, 1996, all of which are incorporated herein by reference in their entirety.

### Composition Use

The present invention also relates to a method of combating denture sores in the mouth of a denture wearer in need thereof, by mixing an anti-ulcer agent with a denture adhesive composition comprising at least 15% of the mixture and providing the mixture to the denture wearer with instructions to use for denture sores. The adhesive compositions may be in the form of a powder, cream, paste, liquid, aerosol, and/or wafer. The subject in need thereof applies the above composition, in any form, to the dentures of a denture wearer in need thereof, and/or to the oral cavity, and/or to the palate or ridge of the oral cavity, and thereafter secures the denture to the ridge or palate of the oral cavity. Pastes (also called gels) are squeezed from a tube. Powder forms are sprinkled on a dental prosthesis, moistened and then inserted into the oral cavity. Denture adhesive compositions with a self-supporting layer are thoroughly moistened, then applied to dentures, and thereafter inserted into the oral cavity.

The anti-ulcer agent may be at least one of: Glycyrrhiza extract, xylitol, and cobalamin.

A denture wearer places from about 0.1 grams to 3 grams of any of the compositions described above on the denture. Then the subject inserts the denture into the mouth and presses it into place. After applying this composition, the composition minimizes, inhibits, and reduces pain from denture sores.

Many variations of these are possible without departing from the spirit and scope of the invention.

I claim:

1. A denture adhesive composition that combats denture sores, comprising at least 15% of a denture adhesive mixture and an effective amount of Glycyrrhiza extract.

2. The denture adhesive composition of claim 1 wherein the Glycyrrhiza extract consists of a complex extract resulting from an extraction with a solvent including water.

3. The denture adhesive composition of claim 1 wherein the Glycyrrhiza extract consists of a complex extract resulting from an extraction with a solvent including alcohol.

4. The denture adhesive composition of claim 1 wherein the Glycyrrhiza extract consists of a refined extract consisting of at least 50% glycyrrhizin and/or glycyrrhetinic acid.

5. A denture adhesive composition that combats denture sores, comprising at least 15% of a denture adhesive mixture and an effective amount of xylitol.

6. A denture adhesive composition that combats denture sores, comprising at least 15% of a denture adhesive mixture and an effective amount of cobalamin.

7. The denture adhesive composition of claim 6 wherein the cobalamin is at least one of cyanocobalamin, hydroxocobalamin, and methylcobalamin.

8. The denture adhesive composition of claim 6 wherein the cobalamin is methylcobalamin.

9. A method of combating denture sores in the mouth of a denture wearer in need thereof, by mixing an anti-ulcer agent with a denture adhesive composition comprising at least 15% of the mixture and providing the mixture to the denture wearer with instructions to use for denture sores.

10. The method of claim 9 wherein the anti-ulcer agent comprises an effective amount of at least one of: Glycyrrhiza extract, xylitol, and cobalamin.

11. A method of combating denture sores in the mouth of a denture wearer in need thereof, by applying a mixture of an anti-ulcer agent with a denture adhesive composition comprising at least 15% of the mixture to at least one of the denture or the mucosa and then securing the denture to the oral cavity.

12. The method of claim 11 wherein the anti-ulcer agent comprises an effective amount of at least one of: Glycyrrhiza extract, xylitol, and cobalamin.
